SEQUENCE LISTING (14 sequences)

SEQ ID NO: 1 - protein, 11 aa, Artificial Sequence
  Synthetic protein transduction domain based on rat transcription factor Hph-1
YARVRRRGPRR

SEQ ID NO: 2 - protein, 9 aa, Artificial Sequence
  Synthetic protein transduction domain based on human transcription factor Sim-2
AKAARQAAR

SEQ ID NO: 3 - protein, 11 aa, Artificial Sequence
  Synthetic protein transduction domain based on HIV-1 TAT protein
YGRKKRRQRRR

SEQ ID NO: 4 - protein, 16 aa, Drosophila pseudoobscura
  DOMAIN (1)..(16): Amino acid residues 353 to 368 of Drosophila ANTP protein
RQIKIWFQNRRMKWKK

SEQ ID NO: 5 - protein, 34 aa, human herpesvirus 1
  DOMAIN (1)..(34): Amino acid residues 268 to 301 of VP22 protein in HSV-1
DAATATRGRSAASRPTERPRAPARSASRPRRPVE

SEQ ID NO: 6 - protein, 7 aa, Artificial Sequence
  Synthetic protein transduction domain R7
RRRRRRR

SEQ ID NO: 7 - protein, 26 aa, Artificial Sequence
  Synthetic protein transduction domain MTS
AAVALLPAVLLALLAPAAADQNQLMP

SEQ ID NO: 8 - protein, 21 aa, Artificial Sequence
  Synthetic protein transduction domain Pep-1
KETWWETWWTEWSQPKKKRKV

SEQ ID NO: 9 - protein, 21 aa, Artificial Sequence
  Synthetic protein transduction domain Pep-2
KETWFETWFTEWSQPKKKRKV

SEQ ID NO: 10 - DNA, 33 bp, Homo sapiens
tatgcacgtgttcggaggcgtggaccccgccgc

SEQ ID NO: 11 - DNA, 18 bp, Homo sapiens
cccaaccctaggccagcc

SEQ ID NO: 12 - DNA, 21 bp, Homo sapiens
acgaggttagggacgggaact

SEQ ID NO: 13 - DNA, 1869 bp, Homo sapiens
gcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcac

SEQ ID NO: 14 - DNA, 3739 bp, Mus musculus
gctgatccccctctagcagtccacttcaccaaggtgagcgagtgtccctgctctcccccaccagacacagctctgctggcgaaagtggcagagaggtattgagggtgggtgtcaggagcccaccagtacagctggaaacacccagccactccagctcccggcaacttctcctgactctgccttcagacgagacttggaagacagtcacatctcagcagctcctctgccgttatccagcctgcctctgacaagaacccaatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttgacctcaaaaccaagaaaaggtgggcgggggagggggccaaaaccatgagactgaggctgtgggggcaaggaggcaagtcctacgtgtacctatggaaaccgggcgatgatgtgcctgctatcagggcctctgctccctatctagctgccctcctagatcatatcatctgccttacagctgagaggggtgccaatcccagcctagcccctagttccaacctagccccaagatgaactttccagtcaaagagccctcacaaccagctatacatatctgccttggccactgccaagcagaaagatgacagacaccatcctaatatttactcaacccaaaccctaaaacatgaagagcctgccttggtacattcgtgaactttcaaagttagtcatgcagtcacacatgactgcagtcctactgactcacaccccaaagcactcacccacaacatctggaaccacgggcactatcacacataggtgtatatacagacccttacacagcaacagcactggaaccttcacaattacatccccccaaaccacacaggcataactgatcatacgcagcctcaagcaatgcccaaaatacaagtcagacacagcttgtcagaacacgctcgtgtgcacgtacacacatgcagcccctccactctatctcctgagttccatgaatacacaccgactctccaagatgtaccccacgtctcacttgccactgaccccagttccctacccacaagccccaatccatgcctaagcgtggcccacagaagaacttctcttttatttgggatccaaggcccctggcccccagtgcccatccaataaactgtggtcagctggacaatcaccctgatcagatatgggaacatataagcagacagctgggtttaagatcccagcaggagaaagcggataccaaatgaaagagagtgctagaacaggtgcctcagcactgtctccagcaccccaaattcctgcctgtggttaggagacatccatcagggctctaggcctctcggacccggcccaagaggccagcattctcctggcgaagggctcggtagtcctcacagatcttctccaggttgctcaaagtcttcttgcccatctctgtctcaatctaagaaaacaggatgcacacttcttcagcccctgcaggctgcccctctactgaactcctccctgctcctcctattcccgtaacagcagcctgttccttcccatcactgggcttctgggtatgtccttccctccactccacctaaagcagcaacttctgccatgggctctgggaggcattaggagccgcaagctaaaagccagggctcagagtaggctactggctagcttcaggtcccaggcacagtgggcacgaaggcaaagcctctagctgttagttgtctggtttcaaagactctcagcgcaaaacaaggaactatcccctggcctgtctccattccccttaccagtcccaggtctcacctgctcctcaagatctcgaacttccctcatgatagtgcctgtgtcctcaatggtctggatgagctgactgcaattctggagacagcaagaatacaaggcttgcacctatgctggccctctccagccaacccaccaggcacatggctcccctcacctcatgcagggcagctaggtacttgtaggctttccgaacagcatcatccttcttagcatcctgataagacaaaggggatctccgagatatcagcaagccattcccccttttccactactctatgcccctataagaccaccctttactagtactttgccttcatcctccacagagcaaagctaggccccaagcaacagtgcacctaaaggactcacagaggggcaggcaacaactcagtcccgcctccaccctcccggaggccagcctgctccataccttgaacacaagctcatcagtcactgcaaatgtccggtcgagcttcccagagagagagttgatttccttctgcagttcctttgtgtccgacaagatctggtagaaaccagggtaactatcagtgcacatcttgggcaaggtagctgatcagtgataacactcacgtgcctatacttacatccagtcagggcccatgtcgctgtgttggggtgactattatgtgttggagtgtgcctgaacagctctgcctagtagtgagcataaagtccctgtgt

(19) United States
(12) Patent Application Publication
(10) Pub. No.: US 2010/0173840 A1
(43) Pub. Date: Jul. 8, 2010

(54) Pharmaceutical Composition for Treating Autoimmune, Allergic and Inflammatory Diseases and Delivery Method Thereof

(76) Inventors: Seung Kyou Lee, Gyeonggi-do (KR); Sang Kyou Lee, Seoul (KR)

Correspondence Address: QUARLES & BRADY LLP, 411 E. WISCONSIN AVENUE, SUITE 2040, MILWAUKEE, WI, 53202-4497, US

(21) Appl. No.: 12/306,036

(22) PCT Filed: Jun. 29, 2007
(86) PCT No.: PCT/KR07/03188

§ 371 Date: Sep. 9, 2009

(30) Foreign Application Priority Data
Jun. 30, 2006 (KR) 10-2006-0061121

(51) Int. Cl. A61K 38/17; A61P 37/06; C12N 5/0783

(52) U.S. Cl. 514/12; 435/325; 435/375

(57) ABSTRACT

Disclosed herein are a novel pharmaceutical composition for inhibiting autoimmune diseases, allergic diseases and inflammatory diseases, which contains a conjugate of Foxp3 and PTD (protein transduction domain), and a delivery method thereof. According to the disclosed invention, Foxp3-PTD treats and inhibits autoimmune diseases by effectively inhibiting the activation of T cells in a mouse autoimmune disease model.

### TECHNICAL FIELD

The present invention relates to a novel pharmaceutical composition for inhibiting autoimmune diseases, allergic diseases and inflammatory diseases, which contains conjugate of Foxp3 and PTD (protein transduction domain), and to a delivery method thereof.

### BACKGROUND ART

Foxp3 is a transcriptional factor, which is present mainly in regulatory T cells derived from the thymus and is present in cells having a CD4+ CD25+ marker antigen. The Foxp3-expressing regulatory T cells act as suppressor T cells that inhibit the IL-2 production and proliferation of T cells, which can potentially induce autoimmune diseases, among Foxp3-non-expressing CD4+ CD25− T cells, which have low antigen affinity in antigen recognition of the Foxp3-expressing T cells and, at the same time, are derived from the thymus. Also, it is known that, through the Foxp3-expressing regulatory T cells and the cell-cell contact of the T cells, Foxp3 performs a function of inhibiting the transcription of not only IL-2 in CD25− T cells, but also IL-4, IFN-gamma and the like, which are influenced by transcriptional factor NFAT.

In order to artificially construct such Foxp3-expresssing T cells and apply them clinically, mouse Foxp3-overexpressing T cells with retrovirus vectors have been successfully used in various applications, including mouse autoimmune disease model EAE (experimental autoimmune encephlaomyelitis), type 1 diabetes models with IBD (inflammatory bowel disease) mice and NOD (non-obese diabetic) mice, and organ transplantation rejection. Also, there have been attempts to apply self-antigen specific T cell clones of human Foxp3-expressing CD4 T cells for cell therapy by treating the clones with a high concentration of IL-2 cytokine and a combination of anti-CD3 and anti-CD28 antibodies to increase the number thereof.

Foxp3 is known to be a lineage marker for regulatory T cells derived from the thymus. It is known that amino acid mutations occurring in the DNA-binding domain of Foxp3 and mutations in the leucine zipper domain of Foxp3 cause the IPEX (immune pathology/polyendocrinopathy/enteropathy/X-linked) syndrome in humans to cause especially eczema (dermatitis) and type 1 diabetes in 6-9 years old children or lead to death due to genetic autoimmune diseases before the age of 2. 70% of IPEX patients are known to be attributable to such Foxp3 mutations.

Methods for delivering macromolecules into cells in vitro include electroporation, membrane fusion with liposomes, high velocity bombardment with DNA-coated microprojectiles, incubation with calcium-phosphate-DNA precipitate, DEAE-dextran mediated transfection, infection with modified viral nucleic acids, and direct micro-injection into single cells. Also, methods for delivering macromolecules into cells in vivo and in vitro using nanoparticles have recently been attempted, but still remain in the early stages of development in technical terms and in view of clinical effects. Such methods can typically deliver macromolecules into only a fraction of the target cells, and the time and efficiency for delivering the macromolecules into cells have not yet reached clinically applicable levels. Moreover, these methods can cause undesirable damage to large numbers of cells other than the target cells.

Thus, general methods for delivering biologically active macromolecules, both in vivo and in vitro, without damaging cells, have been needed [see L. A. Sternson, Ann. N.Y. Acad. Sci., 57, 19-21 (1987)]. For example, chemical addition of a lipopeptide [see P. Hoffmann et al. Immunobiol., 177, 158-170(1988)] or the use of a basic polymer such as polylysine or polyarginine [see W-C. Chen et al., Proc. Natl. Acad. Sci., USA, 75, 1872-1876(1978)] have been suggested, but have not yet been verified. Folic acid has been used as a transporter [see C. P. Leamon and Low, Proc. Natl. Acd. Sci., USA, 88, 5572-5576(1991)], and it was reported to be delivered as folate conjugates into cells, but whether it is delivered to the cytoplasm has not yet been verified. Also, Pseudomonas exotoxin has been used as a transporter [see T. I. Prior et al., Cell, 64, 1017-1023(1991)]. However, the effect of this system on the intracellular delivery of biologically active target materials, and the basic medical and clinical applicability thereof, are not clear. Thus, methods capable of more safely and effectively delivering biologically active materials into the cytoplasm or nucleus of living cells are continually needed.

In addition to the intracellular delivery of macromolecules such as proteins, the intracellular delivery of DNA and/or RNA in vivo or in vitro is also considered to be one of the essential techniques required in the field of biotechnology and applied medical science. The intracellular delivery of DNA and/or RNA plays a decisive role in gene therapies, in basic studies on identifying the function of a protein encoded by the gene in vivo and in vitro, and in developing novel therapeutic agents using DNA and/or RNA. However, since DNA/RNA cannot permeate the cell membrane efficiently, the solution of this problem is one of the biggest problems to be solved in the basic and clinical genetic research fields.

For this reason, liposomes, nanoparticles, viral vectors, etc. have been developed to deliver DNA and/or RNA into cells in vitro and in vivo, and the possibilities of the use thereof have been examined and investigated. However, these vectors have numerous problems to be improved with respect to their efficacy and side effects. In particular, in the case of using liposomes, since there are serious problems of side effects on cells and cytotoxicity, their application has been limited to basic researches with cell lines. In the case of nanoparticles, they have been receiving attention these days, but additional studies on the decomposition of carrier particles in vivo, the in vivo degradation and delivery efficiency of carrier particles, and in vivo immune responses to the carrier particles, are required. In the case of using retroviruses, which are currently important in basic research and in view of clinical effects, there are problems in that the preparation of high-titer retrovirus vectors encounters limitations, and these retroviruses do not infect non-proliferating cells. Adenovirus or adeno-associated virus vectors also have a very limited clinical application. Furthermore, in these two types of viral vectors, in vivo immune responses to the remaining viral proteins are induced, and thus their therapeutic effects are doubtful. Therefore, a novel intracellular delivery method, which is more effective in delivering DNA and/or RNA into cells in vivo or in vitro and has reduced side effects, is continually needed.

Meanwhile, proteins for medical and pharmaceutical purposes, which regulate many physiological phenomena in vivo, have been constructed in the form of recombinant proteins produced in bacteria such as E. coli and have been used to date for the treatment of various diseases. However, because the proteins for medical and pharmaceutical purposes, which are produced in bacteria, are very inefficient in their substantial structure and function as compared to natural proteins produced in cells in vivo, there have been attempts to produce proteins in yeasts, insect cells or animal cells, or to refold the recombinant proteins produced in bacteria, or to produce the proteins using transgenic animals. However, these methods have problems in that they require many molecular cell biological intermediate steps, have a very low yield and are not cost-effective. Thus, it is thought that economically and easily converting recombinant proteins, produced in bacteria, into proteins having the function and structure of natural proteins, will play a decisive role in the development of novel protein drugs for the diagnosis, prevention and treatment of diseases.

Several PTDs (protein transduction domains) have been suggested as a result of such several important basic research and clinical demands. Among them, a Tat protein, which is the transcription factor of human immunodeficiency virus-1 (HIV-1), has been most frequently studied [see Schwarze S R et al, 3:285(5433):1569-1572, 1999 Science]. The Tat protein was found to more effectively permeate the cell membrane, when it comprises amino acids 47 to 57 (YGRKKRRQRRR), on which positively charged amino acids are concentrated, compared to when it is in a full-length form consisting of 86 amino acids (see Fawell S. et al., Proc. Natl. Acad. Sci. USA 91:664-668(1994)). Other examples of PTDs are amino acids 267 to 300 of a VP22 protein of Herpes

Simplex Virus type 1 (HSV-1) (see Elliott G. et al., Cell 88:223-233(1997)), amino acids 339 to 355 of an Antennapedia (ANTP) protein of Drosophila (see Schwarze S. R. et al., Trends Pharmacol Sci. 21:45-48(2000)), and the like. By comparison between the amino acid sequences of said PTDs, if the PTDs abundantly contain lysine and arginine, and in particular, the arginine was considered to play an important role in the intracellular delivery of drugs. This fact was demonstrated because even artificial peptides consisting of positively charged amino acids showed the effect of delivering drugs (see Laus R. et al., Nature Biotechnol. 18:1269-1272(2000)).

MTS, a novel PTD having features different from those of the above-mentioned prior PTD, was synthesized and constructed [see DaeWoong Jo et al., Nat. Biotech. Vol. 19, 2001], and the amino acid sequence thereof was synthesized based on the amino acid sequence of the signal peptide of FGF (fibroblast growth factor).

In previous studies, the present inventors found Hph-1-BTM (SEQ ID NO: 1) in a rat protein transcription factor and Sim-2-BTM (SEQ ID NO: 2) in a human transcription factor, and found that, especially in the case of Hph-1-PTD, these amino acid sequences were completely conserved in about 20 human proteins including human protein Hph-1. Also, the present inventors constructed a novel protein drug, consisting of Hph-1-PTD fused with the cytoplasm domain of membrane protein CTLA-4 that inhibits the activation of cells, and found that the fusion protein effectively inhibits the activation of T cells, and very effectively treats inflammatory immune disease asthma, when it is delivered through the airway [see Choi J M, et al, 2006, Nat Med, May; 12(5): 574-579].

With regard to mechanisms of delivering macromolecules into cells using such PTDs, two hypotheses exist. The first is that PTDs break the cell membrane to deliver macromolecules into cells. The second one is that PTDs use a portion of the cell membrane to form new vesicles, which can deliver macromolecules into cells and release the macromolecules in the cells. In addition, there is also a supposition that PTDs allowing the intracellular delivery of macromolecules can form new channels in the cell membrane, because they have structural features therein, even though they are small-size peptides (see Becker-Hapak M. et al., Methods 24(3):247-256 (2001)).

### DISCLOSURE

### Technical Problem

It is an object of the present invention to provide a novel pharmaceutical composition containing a conjugate of Foxp3 and PTD.

Another object of the present invention is to provide a method of effectively inhibiting autoimmune diseases, allergic diseases and inflammatory diseases using the novel pharmaceutical composition.

### Technical Solution

The present inventors have prepared a conjugate of Mph-1-PTD and Foxp3 for efficient intracellular delivery of the Foxp3 protein, and found that a novel pharmaceutical composition containing the conjugate effectively inhibits the activation of T cells and shows excellent therapeutic effects in animal models of inflammatory immune diseases, asthma and rheumatoid arthritis, thereby completing the present invention.

### Advantageous Effects

The inventive pharmaceutical composition containing the Foxp3-PTD conjugate can effectively inhibit the onset of autoimmune diseases, allergic diseases and inflammatory diseases.

### DESCRIPTION OF DRAWINGS

FIG. 1 shows the structure of a pRSET-B-H2FX3 vector according to the present invention, in which Hph-1 and Foxp3-encoding DNA are inserted in a pRSET-B vector.

FIG. 2 is a photograph showing the expression of the inventive Hph-1-Foxp3 (Hph-1-Hph-1-Foxp3) protein, which was finally purified with elution buffer and stained with Coomassie blue.

FIG. 3 depicts the results of intracellular delivery of Hph-1-Foxp3 and shows that the Foxp3 protein entered cells in a concentration-dependent manner, when the cells were treated with various concentrations of the Foxp3 protein.

FIG. 4 depicts the effect of Hph-1-Foxp3 as a repressive transcription factor and shows that the activity of luciferase, transfected and expressed in cells, was decreased with an increase in the concentration of the Hph-1-Foxp3 protein.

FIG. 5 depicts the IL-2 expression inhibitory effect of Hph-1-Foxp3 and shows ELISA analysis results indicating that the expression level of IL-2 was decreased with an increase in the concentration of Hph-1-Foxp3.

FIG. 6 depicts the IL-2 expression inhibitory effect of Tat-Foxp3 and shows ELISA analysis results indicating that the expression level of IL-2 was decreased with an increase in the concentration of Tat-Foxp3.

FIG. 7 depicts the inhibitory effect of Hph-1-Foxp3 on Jurkat T cell proliferation and shows that the inhibition of cell proliferation of Jurkat T cells treated with Hph-1-Foxp3 was increased with an increase in the concentration of Hph-1-Foxp3.

FIG. 8 depicts the inflammation inhibitory effect of Hph-1-Foxp3 and is a graphic diagram showing the number of lymphocytes in BAL fluid.

FIG. 9 depicts the inflammation inhibitory effect of Hph-1-Foxp3 and shows airway resistance measured as Penh value.

FIG. 10 depicts the therapeutic effect of Hph-1-Foxp3 in rheumatoid arthritis-induced animals and shows clinical scores.

FIG. 11 depicts the therapeutic effect of Hph-1-Foxp3 in rheumatoid arthritis-induced animals and shows pathological findings.

FIG. 12 depicts the contact inhibition effect of CD4+ CD25− T cells transduced with Hph-1-Foxp3 and shows CD4+ CD25− T cells separated using MACS.

FIG. 13 depicts the contact inhibition effect of CD4+ CD25− T cells transduced with Hph-1-Foxp3 and shows the clinical score-reducing effect of the Hph-1-Foxp3-transduced cells in rheumatoid arthritis-induced animals.

FIG. 14 depicts the contact inhibition effect of CD4+ CD25− T cells transduced with Hph-1-Foxp3 and shows the inflammation-mediating cytokine inhibitory effect of the Hph-1-Foxp3-transduced cells in rheumatoid arthritis-induced animals.

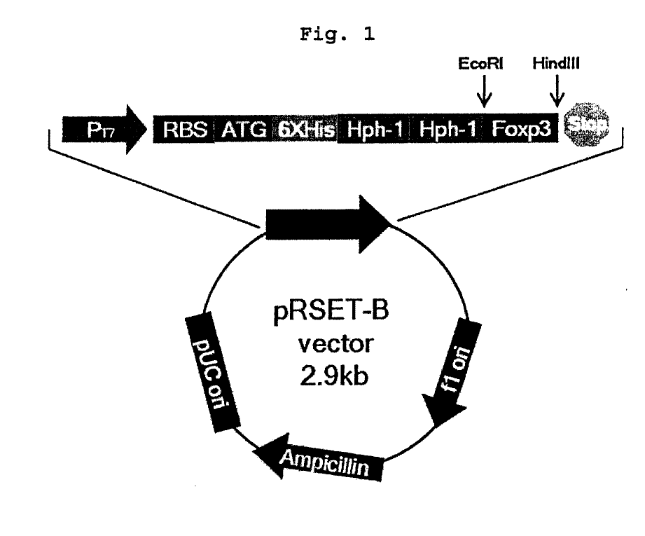

### MODE FOR INVENTION

To achieve the above objects, the present invention provides a conjugate of peptide PTD (protein transduction domain) and Foxp3. The conjugate according to the present invention can be prepared by fusing a PTD-encoding nucleotide sequence with a Foxp3 gene by cloning.

According to the present invention, Foxp3 easily permeates the cell membrane due to the intracellular penetration and delivery effects of PTD so as to be delivered into cells at lesion sites. As a result, the isolated PTD-Foxp3 fusion protein shows the effects of inhibiting, preventing and treating diseases. It was demonstrated that PTD used in the present invention has a very excellent ability to deliver proteins, peptides and chemical compounds into the body through the skin, the eye or the airway, and thus, when it is provided as a conjugate with a drug, it can be delivered to local sites in vivo through various routes.

As PTDs in the present invention, peptides of SEQ ID NO: and SEQ ID NO: 2 below, developed and filed for patent protection by the present inventors, were constructed by a solid phase synthesis method and used in the present invention, but it is to be understood that other kinds of PTDs may be used according to the target site to which a drug is to be delivered, and the kind of linker used. PTD preferably consists of 3-30 amino acids, at least 30% of which are arginines.

Hph-1-BTM:

(SEQ ID NO: 1)

Tyr Ala Arg Val Arg Arg Arg Gly Pro Arg Arg;
Sim-2-BTM:

(SEQ ID NO: 2)

Ala Lys Ala Ala Arg Gln Ala Ala Arg;
HIV-1 TAT:

(SEQ ID NO: 3)

YGRKKRRQRRR;
ANTP:

(SEQ ID NO: 4)

RQIKIWFQNRRMKWKK;
Vp22:

(SEQ ID NO: 5)

DAATATRGRSAASRPTERPRAPARSASRPRRPVE;
R7:

(SEQ ID NO: 6)

RRRRRRR;
MTS:

(SEQ ID NO: 7)

AAVALLPAVLLALLAPAAADQNQLMP;
Pep-1:

(SEQ ID NO: 8)

KETWWETWWTEWSQPKKKRKV;
and
Pep-2:

(SEQ ID NO: 9)

KETWFETWFTEWSQPKKKRKV.

The inventive pharmaceutical composition containing the Foxp3/PTD conjugate can effectively treat: autoimmune diseases, including Crohn disease, rheumatoid arthritis, osteoarthritis, reactive arthritis, psoriatic arthritis, hay fever, atopy, multiple sclerosis, Sjogren's syndrome, sarcoidosis, insulin-dependent diabetes mellitus, autoimmune thyroiditis, ankylosing spondylitis, and scleroderma; allergic diseases, including asthma, contact dermatitis, allergic rhinitis, allergic bronchitis, erythema nodosum, and allergic conjunctivitis; and inflammatory diseases.

Hereinafter, the present invention will be described in further detail with reference to examples. It is to be understood, however, that these examples are illustrative only and are not to be construed to limit the scope of the present invention. The contents of literature cited herein are incorporated herein by reference.

### Examples

### Example 1

### Preparation of Expression Vector Comprising Foxp3

In order to bind a base sequence (5′-TATGCACGTGTTCGGAGGCGTGGACCCCGCCGC-3′) (SEQ ID NO: 10) encoding a peptide, consisting of amino acids ranging from amino acid tyrosine at the 850^{th} position to amino acid arginine at the 860^{th} position from the N-terminus of human transcription factor Hph-1 (GenBank accession No.: NP 004417.2), to Foxp3 (provided by Shimon Sakaguchi, Kyoto University, GenBank accession no. NM 054039, SEQ ID NO: 13), the following base sequences and primers were synthesized: a base sequence (5′-GAATTC-3′) corresponding to restriction enzyme EcoRI for cloning into a pRSETB vector (Invitrogen, cat. #V351-20) having two repeats of a base sequence ranging from amino acid tyrosine at the 850^{th} position to amino acid arginine at the 860^{th} position from the N-terminus of Hph-1; a base sequence (5′-CCCAACCCTAGGCCAGCC-3′; SEQ ID NO: 11) corresponding to the 5°-terminus of the Foxp3 base sequence; a base sequence (5′-ACGAGGTTAGGGACGGGAACT-3′; SEQ ID NO: 12) corresponding to the 3′-terminus of the Foxp3 base sequence; and a primer (5′-AAGCTT-3′) corresponding to restriction enzyme HindIII for cloning. PCR was performed using, as a template, pGEX (Shimon Sakaguchi, Kyoto University) containing a full-length gene, with pfu turbo DNA polymerase (Stratagene, cat. #600252-51).

The PCR reaction product was digested with restriction enzymes EcoRI and HindIII and purified with the Quiaquick PCR purification kit (QIAGEN, cat. #28104). Then, the purified product was cloned into the EcoRI/HindIII site of pRSETB (Invitrogen, Cat. No. V351-20) purified by gel extraction, thus preparing a recombinant expression vector, which was named “pHph-1-Foxp3”.

### Example 2

### Expression and Purification of E. coli Transformant

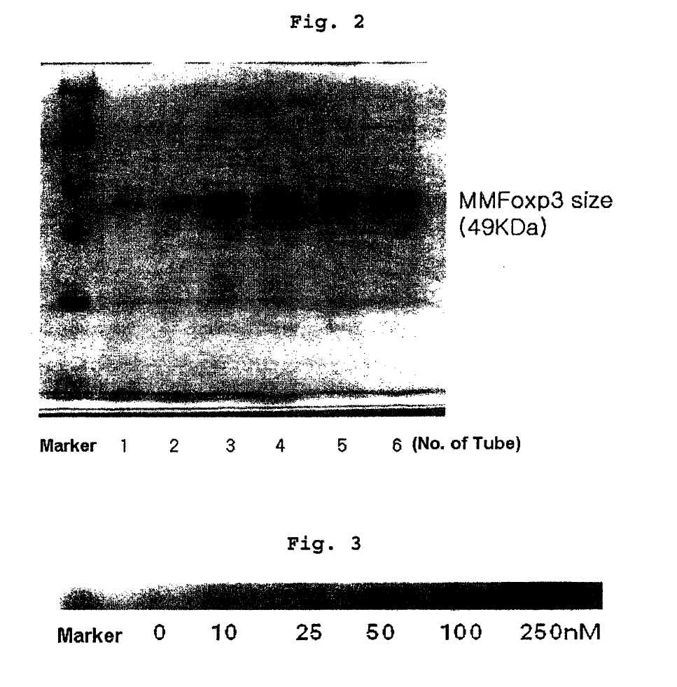

E. coli BL21-Star (Stratagene) was transformed with the expression vector pHph-1-Foxp3 prepared in Example 1, by heat shock transformation, and the transformed E. coli cells were inoculated into 4 ml of LB medium (BD Biosciences) and precultured with stirring at 37° C. for 14 hours. Then, the preculture medium was 250 ml of LB medium (containing 10 g/l casein pancreatic digest, 5 g/l yeast extract and 10 g/l sodium chloride) and cultured at 37° C. for 3 hours. Then, 1 mM IPTG (GibcoBRL cat. #15529-019) was added thereto, and the culture medium was incubated at 37° C. for 4 hours to induce the expression of a fusion protein. The culture medium was centrifuged at 4° C. and 6,000 rpm for 20 minutes to remove the supernatant while leaving only pellets. The pellets were dissolved in 10 ml of buffer solution 1 (100 mM NaH₂PO₄, 10 mM Tris-Cl, 6 M GuHCl, pH 8.0). Then, a process of sonicating the pellet solution on ice for 6 seconds at an intensity of 300 W with an ultrasonic disruptor (Heat systems, ultrasonic processor XL) and cooling the solution for 6 seconds was repeated until the cumulative time of ultrasonication was 5 minutes, such that the pellets were completely dissolved to make the solution clear. The lysate was centrifuged at 4° C. and 12,000 rpm for 20 minutes to remove E. coli fragments and isolate only a pure lysate. 0.5 ml of 50% Ni²⁺-NTA agarose slurry (Qiagen, cat #30230) was added to the isolated solution and stirred at room temperature and 200 rpm for one hour to bind the fusion protein to the Ni²⁺-NTA agarose. The mixed solution was then allowed to flow through a 0.8×4 cm chromatography column (BioRad, cat. #731-1550). The resulting material was washed two times with 10 ml of buffer 2 (100 mM NaH₂PO₄, 10 mM Tris-Cl, 4M GuHCl, pH 6.3) each time, and the fusion protein was fractionated with 1 ml of buffer solution 3 (100 mM NaH₂PO₄, 10 mM Tris-Cl, 2M GuHCl, pH 4.5) and desalted with the PD-10 desalting column (Amersham Pharmacia Biotech., cat. #17-0851-01). The isolated and purified PTD-Foxp3 fusion protein was electrophoresed on SDS-PAGE and visualized by Coomassie blue staining (see FIGS. 2 and 3).

### Example 3

### Effect of PTD-Foxp3 as Repressive Transcription Factor

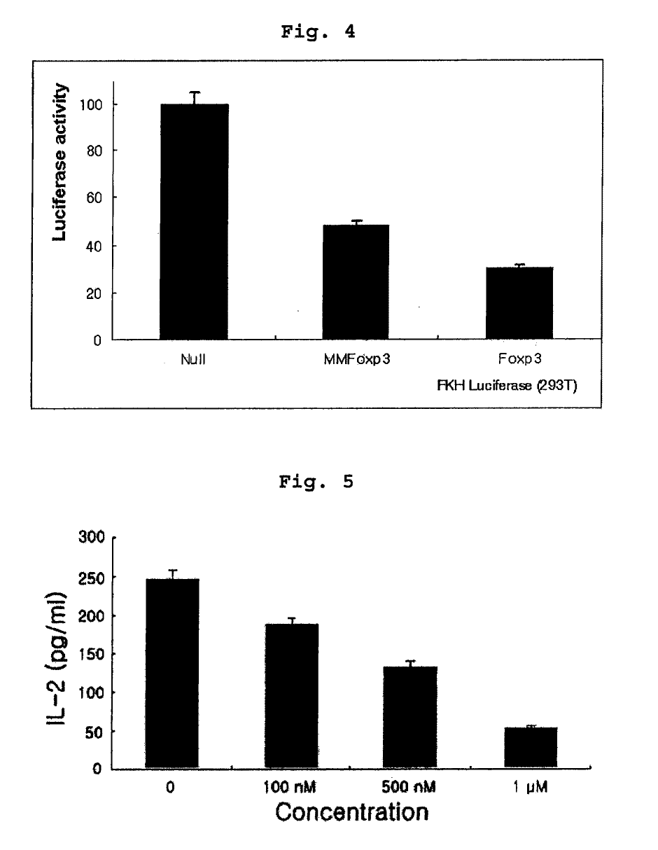

The function of Foxp3 is known to occur, because forkhead DNA binding domains present in Foxp3 bind to forkhead binding sites present in the genome so as to act to inhibit gene transcription. 293 T-cells were transfected with an FKH-luc reporter construct, which contains three successive forkhead DNA binding sites followed by a luciferase gene, together with the Renilla TK plasmid (Promega) for standardizing transfection efficiency. Then, 100 nM PTD-Foxp3 was transduced into the 293 T-cells for 24 hours and, as a result, luciferase activity was inhibited (FIG. 4).

### Example 4

### IL-2 Secretion Inhibitory Effect of Hph-1-Foxp3 in Mouse Jurkat T Cell Line

Foxp3 is known to function to inhibit the production of IL-2 cytokine in T cells. In order to examine whether Hph-1-Foxp3 can perform this function, Hph-1-Foxp3 was transduced into mouse EL-4 T cells. Specifically, in order to induce T-cell activation by a mouse T-cell receptor and T-cell activation by protein CD28 at the same time, a flat-bottom 96-well plate (TPP. Cat No. 92096) was coated with 1 mg/ml of each of anti-CD3 (eBioscience, Cat No. 16-0031) and anti-CD28 (eBioscience, Cat No. 16-0281), and then washed one time with PBS (phosphate buffered saline). Then, 1×10⁵ EL-4 T cells, which were transduced with PTD-Foxp3 at various concentrations of 10 nM, 25 nM and 100 nM, followed by washing three times with PBS, were placed into the previously coated plate, and 10% fetal bovine serum (FBS)-containing RPMI-1640 medium (Biowhittaker, Cat. No. 12-702F) was added thereto. Then, the T cells were stimulated for 24 hours, and the cell lysate was centrifuged. The supernatant was collected and subjected to IL-2 ELISA (R&D system, Cat No. M2000), and the ELISA analysis results are shown in FIG. 5. As shown in FIG. 5, when the EL-4 T cells were treated with Hph-1-Foxp3 at various concentrations of 0, 100 nM, 500 nM and 1 μM, the concentration of IL-2 was decreased in proportion to the concentration of Hph-1-Foxp3.

### Example 5

### IL-2 Secretion Inhibitory Effect of Tat-Foxp3 in Jurkat T Cell Line

In order to confirm whether the same effect also appears in other kinds of PTDs, a recombinant protein was prepared using Tat-PTD, HIV-derived PTD, which is most frequently used for research purposes. The preparation of the expression vector was performed in the same manner as in Example 1, except that a Tat gene (SEQ ID NO: 3) instead of Hph-1 was inserted. The expression and purification of the recombinant protein were carried out in the same manner as in Example 2. To examine the IL-2 secretion inhibitory effect of Tat-Foxp3, cells were treated with the Tat-Foxp3 protein at various concentrations of 0, 100 nM, 500 nM and 1 μM, and the ELISA analysis of IL-2 was carried out in the same manner as in Example 4. As shown in FIG. 5, the secretion of IL-2 was inhibited regardless of the kind of PTD.

### Example 6

### Cell Proliferation Inhibitory Effect of Hph-1-Foxp3 in Jurkat T Cell Line

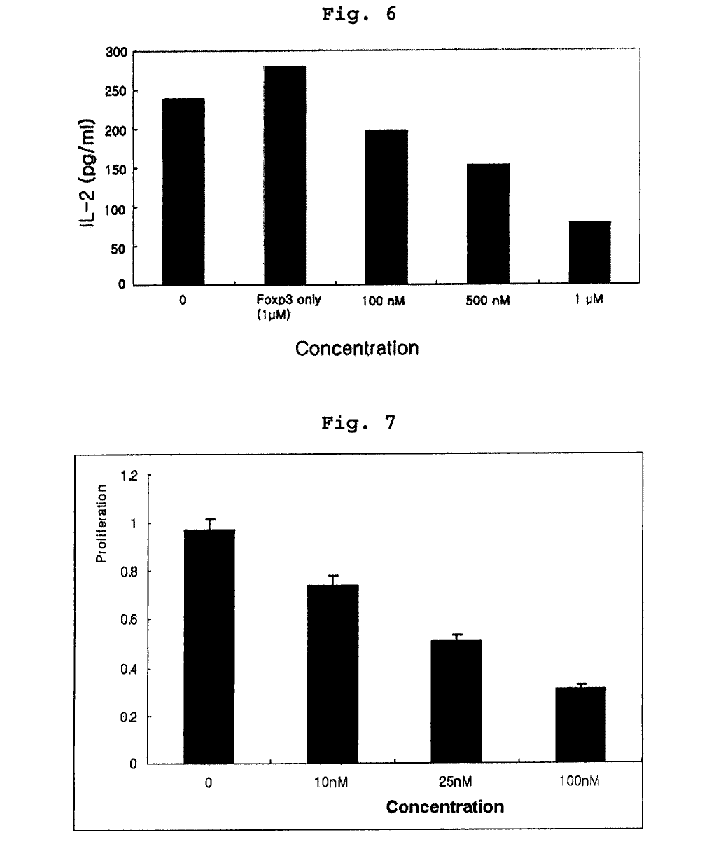

Foxp3 is known to function to inhibit the proliferation and production of T cells. In order to examine whether Hph-1-Foxp3 can perform this function, Hph-1-Foxp3 was transduced into Jurkat T cells. Specifically, in order to induce T-cell activation by a T-cell receptor and T-cell activation by protein CD28 at the same time, a flat-bottom 96-well plate (TPP. Cat No. 92096) was coated with 1 mg/ml of each of anti-CD3 (BD Pharmingen, Cat No. 555329) and anti-CD28 (BD Pharmingen, Cat No. 555725), and then washed one time with PBS (phosphate buffered saline). Then, 1×10⁵ T-cells, which were transduced with HPH-1-Foxp3 at various concentrations of 10 nM, 25 nM and 100 nM, followed by washing three times with PBS, were placed into the previously coated plate, and 10% fetal bovine serum (FBS)-containing RPMI-1640 medium (Biowhittaker, Cat. No. 12-702F) was added thereto. Then, the T cells were stimulated for 24 hours, after which 20 μl of CCK-8 (Cat. No. CK04) was added to each well of the 96-well plate. After that the cell solution in the 96-well plate was incubated in an incubator for cell culture at 37° C. for 2 hours and measured for absorbance at 420 nm with an ELISA reader (Molecular Devices, Cat No. BN02423). The measurement results are shown in FIG. 7. As can be seen in FIG. 7, when the Jurkat T cells were treated with Hph-1-Foxp3 as described above, the cell proliferation of the Jurkat T cells was reduced with an, increase in the concentration of Hph-1-Foxp3.

### Example 7

### Asthma Inhibitory Effect of PTD-Foxp3 in Mice

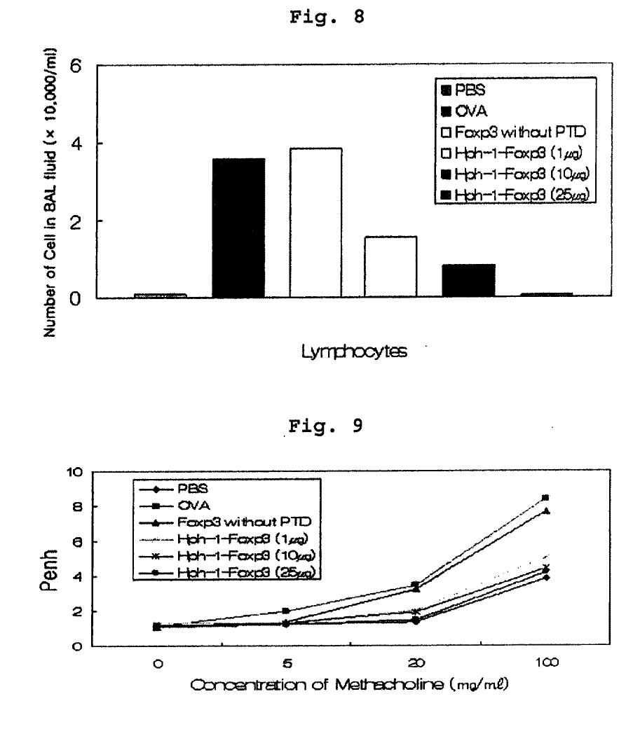

In order to induce asthma, 100 μg of OVA (ovalbumin, Sigma, 9006-59-1) emulsified in aluminum hydroxide (Sigma, 1330-44-5) was injected intra-abdominally into 6-week-old BALB/C mice (Charles River Technology) at day 1 and day 14. Also, at days 15, 16 and 17, 1.5 mg of OVA was injected into the mice to induce asthma. To examine the effect of Foxp-3 in the asthma-induced mice, a novel recombinant protein with mouse Foxp-3 was prepared in this experiment. Although human Foxp3 has an identity of 80% with mouse Foxp3, mouse Hph-1-Foxp3 (SEQ ID NO: 14, Genbank NM054039) was prepared in view of test accuracy. The preparation of the recombinant protein was carried out in the same manner as in Examples 1 and 2. Hph-1-Foxp3 was administered to the asthma-induced mice in amounts of 1 μg, 10 μg and 25 μg through nasal delivery two times a week for 4 weeks. After 4 weeks, the animals were sacrificed, and analyzed for the number of inflammation-mediating cells in BAL fluid (FIG. 8) and airway resistance (FIG. 9).

### Example 8

### Effect of Hph-1-Foxp3 in Rheumatoid Arthritis Model

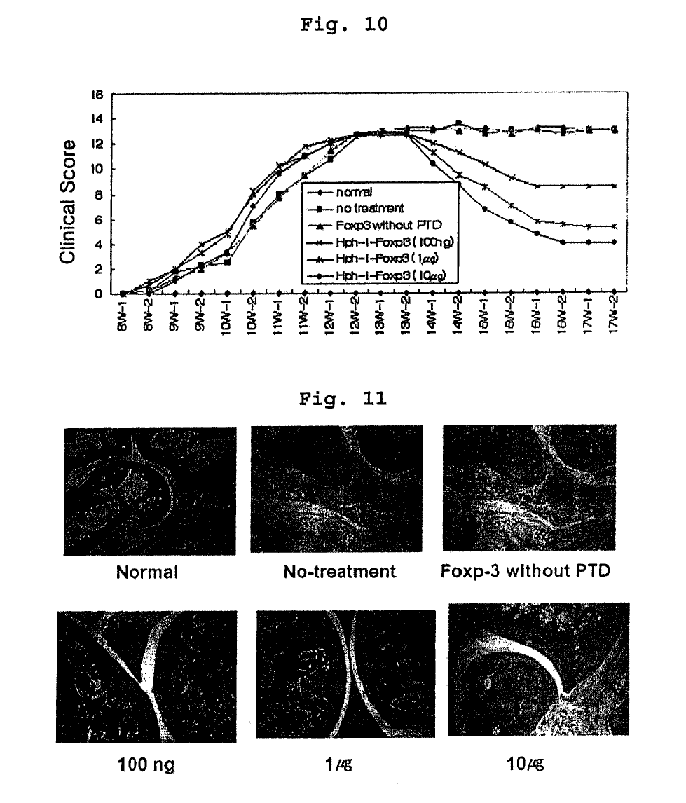

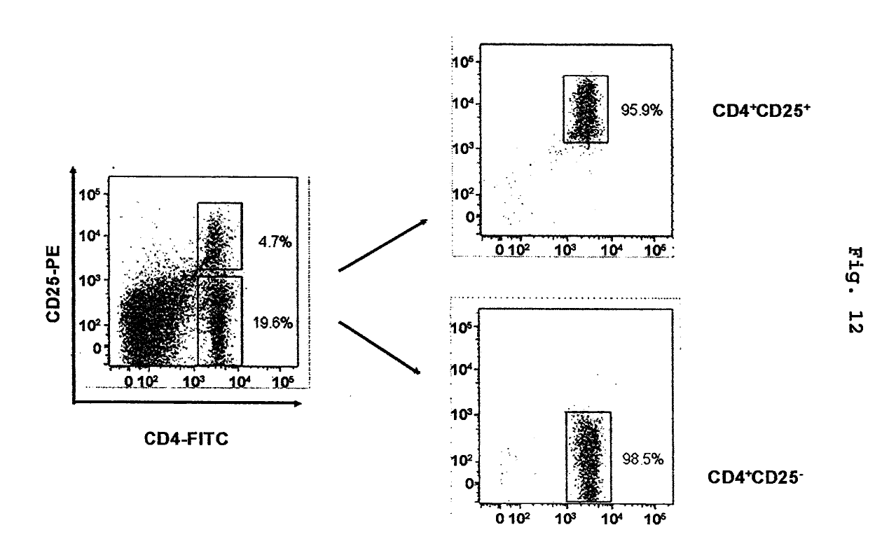

In order to induce rheumatoid arthritis, 100 μg of type II collagen (Sigma, C1188) emulsified in Freund's complete adjuvant (Sigma, F5881) was injected subcutaneously into DBA/1 mice (Central Lab Animal Inc., Korea) on the first day of the experiment and day 14 to immunize the animals. From 4 weeks after the start of immunization, Hph-1-Foxp3 was administered to the animals in amounts of 100 ng, 1 μg and 10 μg two times a week for 4 weeks, and the effect of Hph-1-Foxp3 was verified through clinical score (FIG. 10 and histochemical staining (FIG. 11).

### Example 9

### Therapeutic Effect of CD4+ CD25− T Cells Transduced with Hph-1-Foxp3

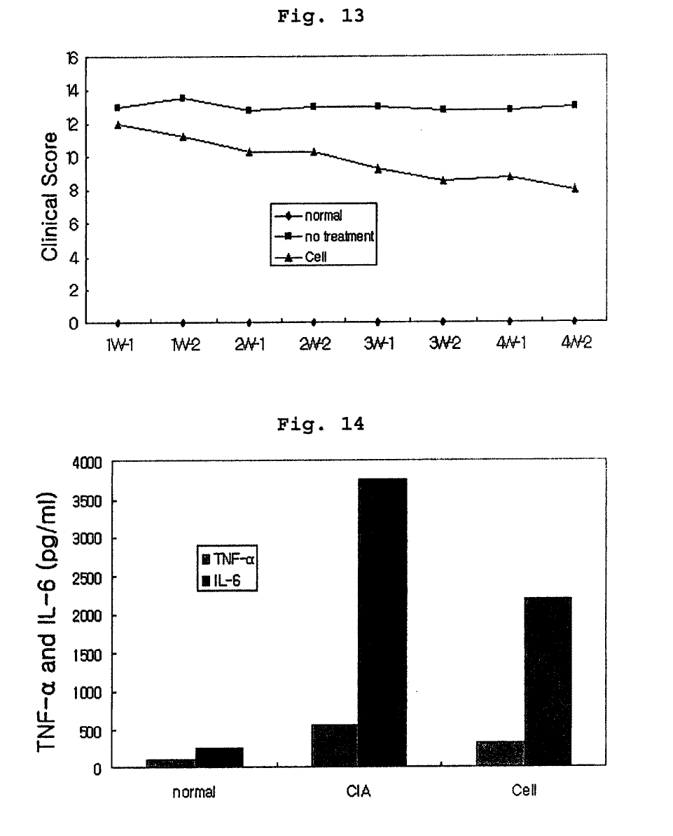

Foxp3-expressing regulatory T cells are known to perform a function of inhibiting the transcription of not only IL-2 in CD25− T cells through cell-cell contact, but also IL-4, IFN-gamma, etc., which are influenced by transcription factor NFAT. For this reason, therapeutic effects through contact inhibition can be shown by transducing Hph-1-Foxp3 into CD4+ CD25− T cells separated from the spleen of mice and treating autoimmune disease-induced mice with the Hph-1-Foxp3-transduced cells. The present inventors first separated the spleen from DBA/1 mice (Central Lab. Animal Inc.) and separated CD4+ CD25(−)(+) T cells from the spleen with MACS (Miltenyi Biotec) using a CD4+ CD25+ isolation kit (Miltenyi Biotec, 130-091-041) (see FIG. 12). The CD4+ CD25− T cells thus separated were treated with Hph-1-Foxp3 at a concentration of 1 μM and cultured for 3 hours. The cultured cells were centrifuged at 1,000 rpm for 3 minutes and washed with PBS (phosphate-buffered saline, 137 mM NaCl, 2.7 mM KCl, 10 mM Na₂HPO₄, 2 mM KH₂PO₄), and this process was repeated three times. The cells thus prepared were suspended in PBS at a concentration of 1×10⁴ cells/100 μl, and mice having rheumatoid arthritis induced as described in Example 8 were injected with 100 μl of the cell suspension slowly for 1 minute through the tail vein. While the mice were monitored for 4 weeks after injection of the cell suspension, the clinical scores of the mice were measured (see FIG. 13). After 4 weeks, TNF-α and IL-6 in the mice's blood were analyzed (see FIG. 14). The analysis results revealed that the CD4+ CD25− T cells transduced with Hph-1-Foxp3 can function as cell immunosuppressive therapeutic agents through contact inhibition.

Although the preferred embodiments of the present invention have been described for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, the inventive pharmaceutical composition containing the Foxp3-PTD conjugate can effectively inhibit the onset of autoimmune diseases, allergic diseases and inflammatory diseases.

1. A pharmaceutical composition for treating autoimmune disease, allergic disease and inflammatory disease, which contains a pharmaceutically effective amount of a Foxp3-PTD conjugate.

2. The pharmaceutical composition of claim 1, wherein the PTD comprises one or more peptides selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

3. The pharmaceutical composition of claim 1, wherein the PTD consists of 5-30 amino acids, at least 30% of which are arginines.

4. The pharmaceutical composition of claim 1, wherein the autoimmune disease is Crohn disease, rheumatoid arthritis, osteoarthritis, reactive arthritis, psoriatic arthritis, hay fever, atopy, multiple sclerosis, Sjogren's syndrome, sarcoidosis, insulin-dependent diabetes mellitus, autoimmune thyroiditis, ankylosing spondylitis, or scleroderma.

5. The pharmaceutical composition of claim 3, wherein the autoimmune disease is Crohn disease, rheumatoid arthritis, osteoarthritis, reactive arthritis, psoriatic arthritis, hay fever, atopy, multiple sclerosis, Sjogren's syndrome, sarcoidosis, insulin-dependent diabetes mellitus, autoimmune thyroiditis, ankylosing spondylitis, or scleroderma.

6. The pharmaceutical composition of claim 1, wherein the allergic disease is asthma, contact dermatitis, allergic rhinitis, allergic bronchitis, erythema nodosum, or allergic conjunctivitis.

7. The pharmaceutical composition of claim 3, wherein the allergic disease is asthma, contact dermatitis, allergic rhinitis, allergic bronchitis, erythema nodosum, or allergic conjunctivitis.

8. The pharmaceutical composition of claim 1, which is administered in vivo by intravenous injection, intraarterial injection, intramuscular injection, subcutaneous injection, intraconjunctival injection, or airway inhalation.

9. T cells having immunosuppressive activity, obtained by treating T cells with a recombinant protein consisting of a Foxp3-PTD conjugate.

10. A method for preparing T cells having immunosuppressive activity, the method comprising treating T cells with a recombinant protein consisting of a Foxp3-PTD conjugate.

11. A pharmaceutical composition for treating autoimmune disease, allergic disease and inflammatory disease, which contains a pharmaceutically effective amount of the T cells of claim 9.
